(19) United States
(12) Patent Application Publication
(10) Pub. No.: US 2021/0275023 A1
(43) Pub. Date: Sep. 9, 2021

(54) HEALTH MONITORING SYSTEM FOR WELLNESS, SAFETY, AND REMOTE CARE USING DIGITAL SENSING TECHNOLOGY

(71) Applicants: Haik Kalantarian, Carnation, WA (US); Artak Kalantarian, Carnation, WA (US); Susan Makarian, Carnation, WA (US); Sergey Baghdasaryan, Yerevan (AM)

(72) Inventors: Haik Kalantarian, Carnation, WA (US); Artak Kalantarian, Carnation, WA (US); Susan Makarian, Carnation, WA (US); Sergey Baghdasaryan, Yerevan (AM)

(73) Assignee: Haik Kalantarian, Carnation, WA (US)

(21) Appl. No.: 16/807,445

(22) Filed: Mar. 3, 2020

(51) Int. Cl. A61B 5/00; A61B 5/11; A61B 5/16; G16H 50/30; G16H 15/00; G06N 20/00

(52) U.S. Cl. CPC A61B 5/0022; A61B 5/1112; A61B 5/165; G06N 20/00; G16H 50/30; G16H 15/00; A61B 5/4815

(57) ABSTRACT

The invention is a system for remote health monitoring, and a framework for deriving behavioral data from wearable sensors for the screening and tracking of disease. The mobile/wearable computing device captures a host of metrics related to an individual's health, behavior, and wellness. Data from these sensors is aggregated, analyzed, and reported to one or more caregivers via a caregiver dashboard made available as a smartphone, web, or smartwatch application. This data consists of time-sensitive notifications such as falls, wandering, and extreme weather alerts, as well as a holistic profile of the individual's wellness including stress, sleep quality, fitness, and longitudinal lifestyle changes. As the mobile system is used in the field, a large-scale database of behavioral data is generated from the userbase, providing a flexible platform for the longitudinal tracking and screening of a variety of mental, neurological, and physiological disorders.

### BACKGROUND

It is estimated that there are over 47 million adults over the age of sixty-five in the United States alone. Many of these individuals live in a state of independence or semi-independence, and benefit from the oversight of one or more caregivers. Examples of caregivers include children, nieces, nephews, grandchildren, siblings, or the staff of care facilities. Despite the best intentions of the caregivers, their means of monitoring the patient is the laborious and intermittent process of manual check-in such as phone calls and emails. Outside these check-in times, many patients may not benefit from any oversight whatsoever.

Existing remote health monitoring solutions are designed for the detection of catastrophic health events such as heart attacks, stroke, or falls, and are not designed as a continuous monitoring and/or reporting tool to decrease the psychological burden of caring for an individual who cannot be accompanied at all hours of the day. Thus, existing platforms for seniors cannot effectively provide continuous real-time feedback and positive assurances of the patient's well-being as informed by multiple disparate sensing modalities including audio, inertial sensors, proximity sensors, GPS signals, and other sources of data.

Furthermore, many remote health monitoring systems cannot effectively leverage the ubiquity of commercial off-the-shelf devices such as smartphones and smartwatches—which are typically associated with a much higher rate of compliance than expensive custom-made devices that represent major barriers to adoption and long-term adherence. What few mobile systems exist for remote care are highly limited in scope. These platforms lack methods of detection of device adherence, falls, location change, sleep quality, and stress of sufficient sophistication to manage the tradeoffs of false positives, latency, and accuracy appropriately, rendering them unsuitable for real-time remote health monitoring.

Lastly, there is a dearth of comprehensive mobile systems to effectively capture complex data from multiple digital sensors, perform signal processing, machine learning, and run pattern recognition to holistically characterize an individual's lifestyle, wellness, and behavior for the screening, tracking, and treatment of disease. Existing platforms do not leverage heuristics such as the rate of interaction with the mobile device, screen time, pacing behavior, sleep habits, and time spent outside the home with conditions such as depression, anxiety, and Parkinson's using statistical learning techniques in a manner that informs the caregiver of the existence and severity of these conditions in real-time.

### SUMMARY

The present invention is a system for the remote health monitoring, designed for but not limited to individuals of limited independence such as the elderly, those with mild cognitive impairment, or children, as well as a framework for deriving behavioral data from mobile/wearable sensors. The platform is designed to increase the safety and wellness of the patient, minimize the physical and psychological burden of caring for another, and providing a flexible platform for the screening and tracking of disease from longitudinal behavioral data.

The system consists of a mobile/wearable device which collects and calculates a variety of health, safety, and wellness data such as their location, environment, fitness levels, heart rate, sleep quality, current activity, if they have fallen, stress levels, behavioral trends, and more. This information is processed and transmitted to a caregiver, who can view and visualize the data and receive real-time notification of potential hazards.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1 illustrates the data flow in which the patient wears a mobile computing device, which collects behavioral data from sensors to be transmitted to a caregiver through a server.

FIG. 2 shows the system architecture of the mobile/wearable device worn by the patient, consisting of a variety of onboard sensors and peripherals.

FIG. 3 shows the major features and general layout of the caregiver dashboard, with notifications displayed at the top and cards in the center with an overview of information associated with the patient's well-being.

FIG. 4 shows an example of a detailed view in the dashboard.

FIG. 5 is an example of a detail view associated with a push notification on the caregiver's device.

FIG. 6 shows the subsystem to detect if the patient has fallen down and may be incapacitated.

FIG. 7 shows the subsystem to detect if the device is worn by the patient.

FIG. 8 shows the subsystem to detect the quality of the patient's sleep.

FIG. 9 shows the subsystem to calculate the patient's stress score.

FIG. 10 shows the subsystem used to characterize internal or external environmental conditions, using a combination of 3^{rd} party weather APIs and a temperature-enabled Bluetooth beacon.

FIG. 11 shows the Machine Learning subsystem, which correlates behavioral data derived from wearable sensors with health outcomes.

FIG. 12 shows a simplified representation of the Machine Learning subsystem.

FIG. 13 shows the subsystem to detect location change.

### DESCRIPTION OF EXAMPLE EMBODIMENT

Note, the following description is presented to enable one of ordinary skill in the art to make and use the proposed techniques. Descriptions of specific embodiments and applications are provided only as examples and various modifications will be readily apparent to those skilled in the art. The general principles described herein may be applied to other embodiments and applications without departing from the scope of the disclosure. Thus, the present disclosure is not to be limited to the embodiments shown but is to be accorded the widest scope consistent with the principles and features described herein. For purpose of clarity, features relating to technical material that is known in the technical fields related to the proposed ideas are not been described in detail.

The terminology used herein is for the purpose of describing particular embodiments only and is not intended to be limiting the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

### System Overview

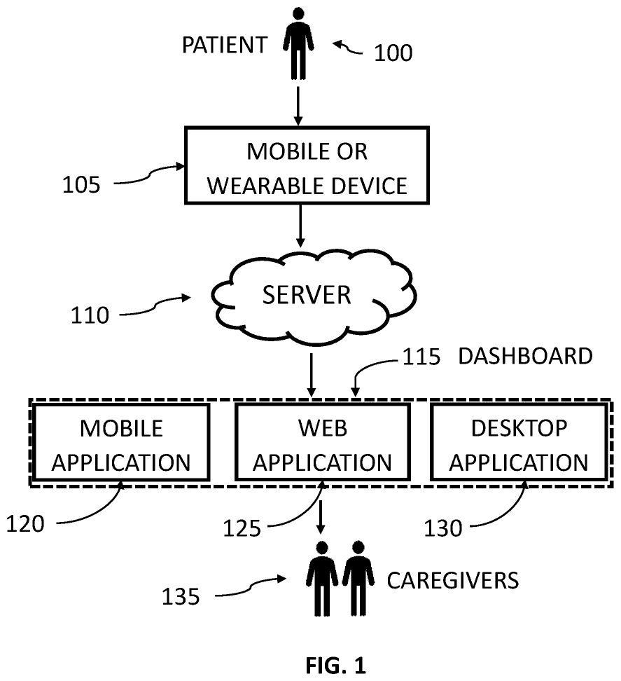

The present invention consists of a framework in which one individual, referred henceforth as the patient 100, can be remotely digitally monitored by one or more other individuals, referred to herein as the caregiver(s) 135. An example embodiment of such a system is shown in FIG. 1. The patient 100 will carry a mobile, digital device on their person 105 that could include but is not limited to: a digital wristband, smartwatch, phone, beacon, or other wearable, which includes sensors and communication capabilities such as cellular, WiFi, or Bluetooth. In other embodiments, the device will not be carried but will be placed in a location of significance to the patient such as a home. Subsystems running on the smart device will detect various behaviors and physiological parameters, and transmit them to the caregiver dashboard 115, optionally through an intermediary database or server 110. The server will aggregate, filter, and further process the data before relaying it to the caregiver dashboard 105.

The caregiver dashboard could consist of software running on a mobile device such as a smartphone 120, a web page or web application, 125, a desktop application 130, or another platform such as a browser plug-in or social media integration. The caregiver dashboard is considered the primary medium through which the caregiver 135 is informed of the patient's health and wellness in this embodiment. The dashboard software receives data from the patient device either directly through a device-to-device communication such as Bluetooth, through the intermediary server 105 using web APIs (WiFi, Cellular) or another form of network communication (e.g. Zigbee, Z-Wave).

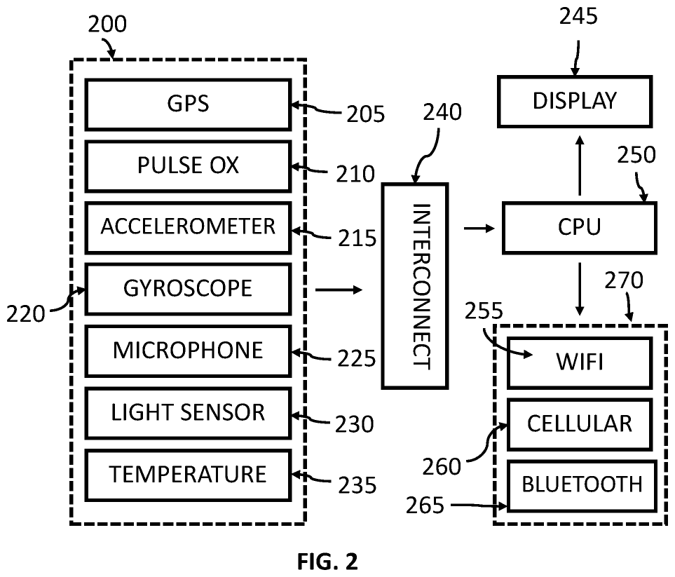

An example embodiment of the mobile or wearable device to be used by the patient 100 for reporting of physiological and behavioral data 105 is shown in FIG. 2. At a minimum, the device should consist of an array of sensors 200, a microprocessor 250, and a communications module 270 to transmit acquired data either directly to the caregiver or indirectly through a web server/database 110. The array of sensors 200 could include but is not limited to the following: GPS, pulse oximeter, accelerometer, gyroscope, magnetometer, microphone, camera, light sensor, humidity sensor, pressure sensor, and a temperature sensor. Additional on-device logic to filter, smooth, and process acquired sensor data could run on the microprocessor 250 and optionally be displayed on the device display 245 similar to a smartwatch. The communications module 270 could include but is not limited to: WiFi 255, cellular 260, Bluetooth 265, or Zigbee. The interconnect 240 would be any internal bus or communication protocol to exchange data between the device firmware and/or software running on the CPU 250 and hardware modules such as the sensor array 200, display 245, communications module 270, and any internal memory. It should be noted that some embodiments of the mobile and wearable device 100 could include off-the-shelf hardware such as an Apple Watch or a mobile phone running Android/iOS software.

As will be detailed later, several behavior and wellness recognition algorithms will run on the CPU 250. However, the term “CPU” is used abstractly, and it is not required that the CPU 250 associated with the patient's mobile or wearable device 105 take the form of a general-purpose microprocessor. The techniques described herein can also be implemented by one or more special-purpose computing devices. The special-purpose computing devices may be hard-wired to perform the techniques, or may include digital electronic devices such as one or more application specific integrated circuits (ASICs) or field programmable gate arrays (FPGAs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, or FPGAs with custom programming. Moreover, it is entirely possible that the CPU 250 merely acquire the data and facilitate the transmission of raw sensor values to the server, 110 upon which the actual processing is to take place.

### Caregiver Dashboard

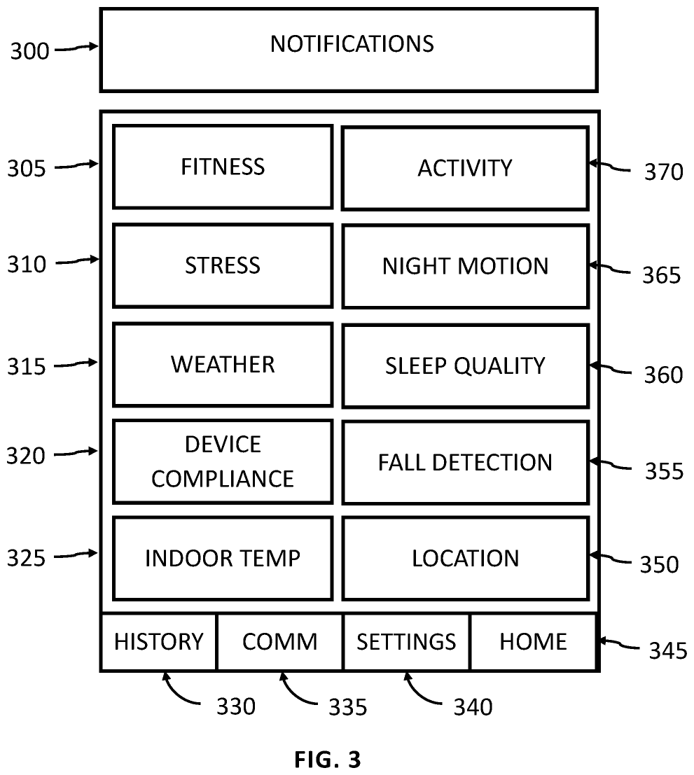

An example embodiment of the caregiver dashboard 115 is shown in FIG. 3. While the dashboard could take many forms, primarily it is the software running on a device used by the caregiver 135 that functions as the primary gateway to receive updates, alerts, and notifications about the well-being of the patient 100. The top of the dashboard is the notification tray 300, where urgent status updates about the health and wellness of the patient are displayed. If the dashboard were manifested in the form of an Android application, this 300 could be the system notification tray which would enable the caregiver 135 to receive updates even when the application was closed. Alternatively, the notification tray could take the form of an SMS notification, email, or audio queue. The Notification Subsystem delivers important, time-sensitive updates to the caregivers with respect to the patient's health. While it is included in REF_Ref31974491\h\*MERGEFORMAT FIG. 3 in this particular embodiment, it may be external to the caregiver dashboard software.

The bottom of the dashboard 115 is the navigation panel, with links to several other interfaces. The history 330 page includes a list of chronological events relevant to the patient's wellness and could include updates about when the patient left the house, when they started driving, or when their location changed. This interface is designed to provide a summary of historic data related to the patient's health and behavior. The communication 335 tab provides the caregiver 135 with a gateway to communicate directly with the patient by means of voice or text. For example, the caregiver can press a button in this interface to initiate a call. The settings tab 340 enables the caregiver 135 to customize which notifications they would like to receive. For example, a caregiver may elect not to receive notifications when the patient's location has changed but may still desire to be notified if the patient has fallen. Furthermore, the caregiver can use this interface to decide how they wish to be notified. For example, they may disable text message notifications but enable mobile push notifications or emails. Moreover, this is the interface through which the caregiver can select the sensitivity of the motion detector 605 to be described hereafter in the Fall Detection module. Additional settings and customizations are also housed within the settings page.

The center of the dashboard 115 consists of a number of cards, which provide brief summaries of one or more aspects of the patient's health and wellbeing that are updated in real-time. We now provide an enumeration of the different cards for this particular embodiment of the proposed system, though it should be understood that other embodiments could feature additional cards, or a subset of the cards described herein. The fitness card 305 displays the number of steps the caregiver has taken during the course of the day. This would be achieved through integration with a platform-dependent API. For example, the Google Fit API could provide such information on Android devices. Alternatively, a standard algorithm for detecting a step from accelerometer 215 data could be employed. As the ability to detect step counts from an accelerometer or platform fitness API is straightforward to those skilled in the art of wearable system design, we do not elaborate further on implementation strategies.

The stress card 310 provides a summary of the estimated stress the patient is experiencing, in the form of a numerical score and visualization. The weather card 315 displays the current weather conditions in the locale of the patient 100, which are highlighted in red if the conditions are unusual or potentially hazardous. The Device Compliance card 320 indicates whether the patient is currently wearing the device. The indoor temperature card 325 shows the temperature in the patient's immediate vicinity, as informed by smart-home integration or a temperature-enabled Bluetooth beacon.

The activity card 370 displays the patient's current activity, which includes but is not limited to: walking, driving, running, or idle. It can also display an alarm informing the caregiver 135 that the patient 100 is in an extended period of inactivity. The night-motion card 365 displays any indication that the patient has been active during typical night-time hours. The sleep-quality card 360 displays a sleep quality score, that summarizes the quality of the patient's sleep on the previous night. The fall detection card 355 displays an alert if the patient has fallen down in a recent period. Lastly, the location card 350 displays a map of the patient's current location. This card can also optionally show an alert indicating to the caregiver that the patient's location has recently changed.

The activity card is also tasked with alerting the caregiver if the patient has been idle for an extended period of time, which is four hours in this particular embodiment. That is, if the patient's hourly step total does not exceed a minimum threshold for four hours in a row, the activity card 305 will display a message to the caregiver alerting them of the patient's prolonged inactivity. Other embodiments could use distance travelled in lieu of step counts. Regardless of the metric being employed, extended inactivity information would also be delivered to the caregiver through the Notification subsystem 300 to be described below.

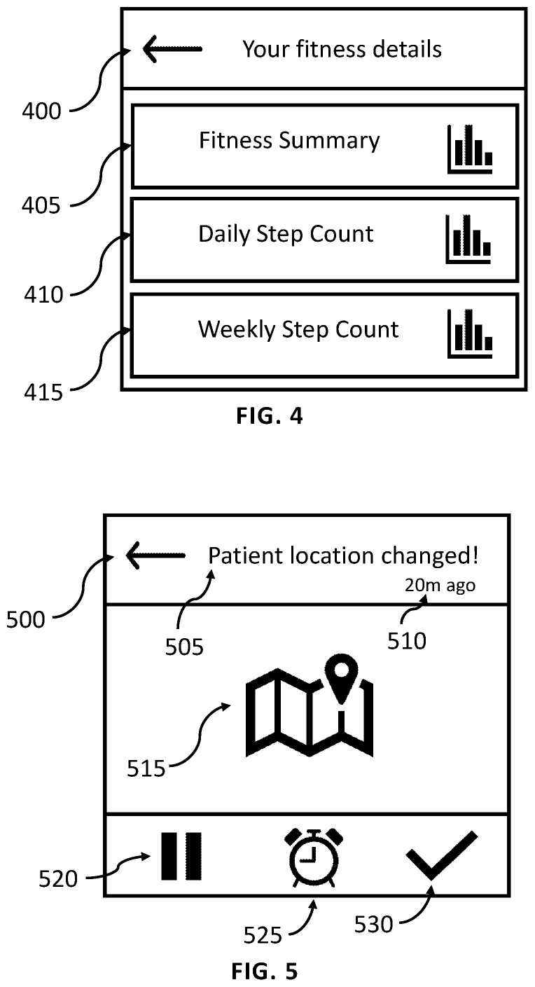

When the caregiver presses, taps, or selects a card in the dashboard, they are presented with a secondary view detailing additional information about that particular activity or event. An example embodiment of such a view is the popup shown in FIG. 4, which is associated with the Fitness card 305. The card features a summary of the status of the patient with respect to that metric 405. Importantly, this summary is context-aware and reflects the user's recent behavior. Additional summaries are shown at the bottom, detailing the step count of the patient 100 over the last day, as well as the last week 415. The card also includes a back button that returns the user to the main dashboard if selected.

On some occasions, the system detects a time-sensitive event and urgently notifies the caregiver through the notification interface 300. An example of such an event is that in which the patient's 100 location has significantly changed in a manner that is unexpected to the caregiver 135. In this embodiment, the appropriate card on the dashboard would be highlighted with a visual cue (e.g. alternative color) to indicate a degree of urgency. Should the caregiver select the card in its alarm state, they would be presented with an alternative detail view. One particular embodiment of such a view is shown in FIG. 5 in which the patient's location has changed. In this scenario, the back button 500 differs from 400 in that dismissing the popup may require an explicit acknowledgement. Furthermore, the card title 505 would be modified to reflect the nature of the event, and a timestamp 510 would detail the time in which the event occurred. The main body of the card 515 would show additional details, which in this embodiment would be a map showing the trajectory of the patient 100.

Almost all time-sensitive events will be associated with a notification delivered through the notification subsystem 300, which includes but is not limited to: text message, email, phone call, or mobile push notification on a smartphone. The caregiver can elect to disable or re-enable such notifications as appropriate using the Pause/Play button at the bottom of the popup 520. When the notification is paused, the dashboard status may continue to be updated. However, the notification subsystem 300 will be disabled for that particular type of notification. Additionally, the caregiver can snooze the notification 525, which will disable it for a selected period of time before it is automatically re-enabled. Lastly, the caregiver 135 can elect to explicitly acknowledge and dismiss the notification using the checkmark button 530. In most cases, doing so will revert the status of the card on the dashboard to the pre-alert state.

### Device Compliance Subsystem

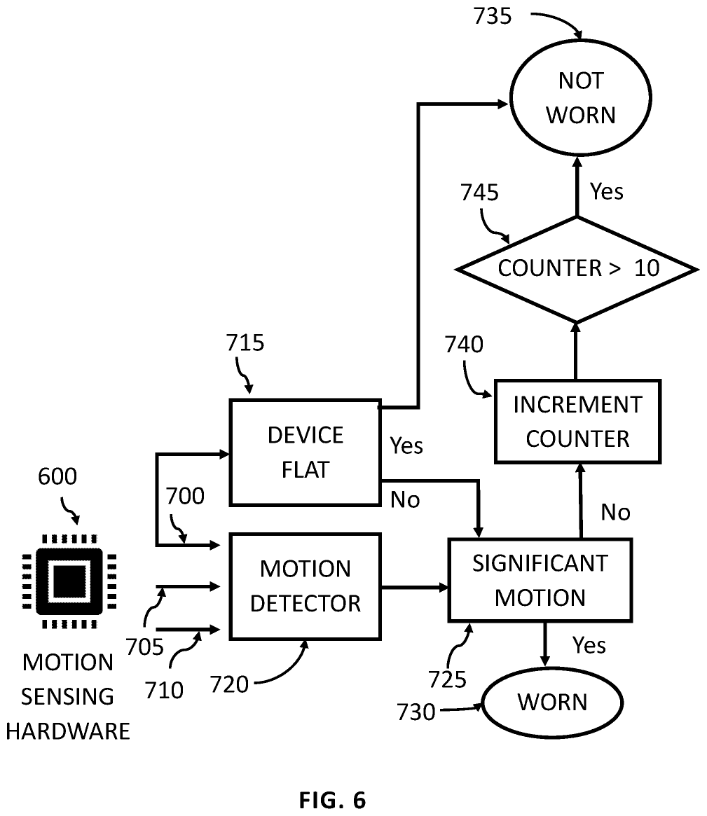

Data about the patient's compliance with the system is determined by the hardware/software subsystem, one embodiment of which is shown in FIG. 6, and is displayed on the caregiver dashboard 115. In the context of this module, “worn”, “used”, and “compliance” are used interchangeably, as are “not worn”, “unused”, and “non-compliance.” The module described herein detects both if the device is worn and/or if the device is in use using the same mechanism.

In this embodiment, the Device Compliance subsystem is in a state of continuous evaluation throughout the device lifetime, running periodically to update the device between the possible states of Worn 730 and Not Worn 735 as necessary. In the case of a state change, this information is sent from the caregiver device to the patient through the foregoing architecture illustrated in FIG. 1. The rate at which the subsystem runs is an ancillary implementation detail that is primarily a function of the accelerometer buffer size; this particular implementation runs at a rate of once per minute, as the buffer can only hold approximately one minute worth of data at a time.

The hardware sensor that provides the foundation for the Device Compliance subsystem is the motion sensing hardware 600. This hardware is a hardware sensor that could comprised of an accelerometer, gyroscope, or magnetometer. It could also be embodied by any other form of motion sensing hardware. In this embodiment, the motion sensing hardware contains a triaxial accelerometer. Three axes of the accelerometer from the motion sensing hardware are sampled periodically, and the data is stored in a hardware or software buffer (queue). The motion detector block 720 reads data from the buffer and calculates the peak intensity of the last n seconds of accelerometer data. The peak intensity is the largest magnitude accelerometer reading in the buffer when interpreted as a vector in 3D space. Given an accelerometer reading of (x, y, z) where x, y, and z are the respective acceleration values in orthogonal directions represented in meters per second, the magnitude could be represented as \sqrt{x²+y²+z²}. However, other representations are also possible—including those that use only a single axis of the accelerometer or gyroscope, or some combination of multiple sensors. Thus, the peak intensity in the last n seconds of data is the accelerometer value in the buffer with the highest magnitude.

The peak intensity point in the accelerometer buffer is sent to the Significant Motion screening block 725, which calculates a Boolean significance value for the buffer of data. This is achieved by comparing the peak intensity with a predefined threshold dependent on the sample rate of the device, but roughly corresponding with the intensity of a phone being jostled around gently in a pocket. If a significant motion is observed, the system state will transition immediately to Worn 730. If no significant motion is revealed 725, a software counter is incremented on the patient device 740. If the value of the counter exceeds some threshold (ten in this particular embodiment) 745, the device will transition to the Not Worn state and the counter is reset. If the value of the counter does not exceed the threshold, this iteration of the subsystem is complete, and the device state does not change. This delay in state transition is to prevent false negatives caused by scenarios in which the device is worn but the user is very still for several minutes. Thus, in this embodiment, the subsystem would need to run ten times (once per minute) and detect no significant motion in each iteration in order to finally transition to the Not Worn state. If at any point the device is worn, the state transitions to Worn immediately and the counter is reset.

The three accelerometer axes from the motion sensing hardware are 700, 705, 710. Significantly, one particular axis 700 would be parallel to the direction of gravity and thus have an absolute value near the gravitational force of 9.8 m/s if the device was placed flat on a surface or table. Comparing the magnitude of acceleration in this axis to the gravitational force can provide a simple heuristic to determine if the device is placed flat, and thus if the system should be in the Not Worn 735 state. Unlike many alternative methods that use angular velocity data reported by the gyroscope in the motion sensing hardware 600, this embodiment determines device orientation using the accelerometer only. This is advantageous because many low-cost motion sensing modules do not contain a gyroscope. If anytime it is found that the device has been placed flat, the system state will transition immediately to Not Worn 735 without waiting for the counter to exceed the delay threshold 745. Thus, this approach quickly accelerates the detection of device non-compliance in this scenario.

### Fall Detection Subsystem

The system is capable of detecting if the patient has fallen down and can instantly alert the caregiver of this emergency event through the dashboard 115 and notification interface 300. As was the case for the Device Compliance subsystem, the Fall Detection subsystem is in a state of continuous evaluation throughout the device lifetime, running periodically to determine if the patient has fallen. The rate at which the subsystem runs is a function of the accelerometer buffer size and desired latency and is considered an ancillary implementation detail. In this embodiment, the algorithm runs every minute.

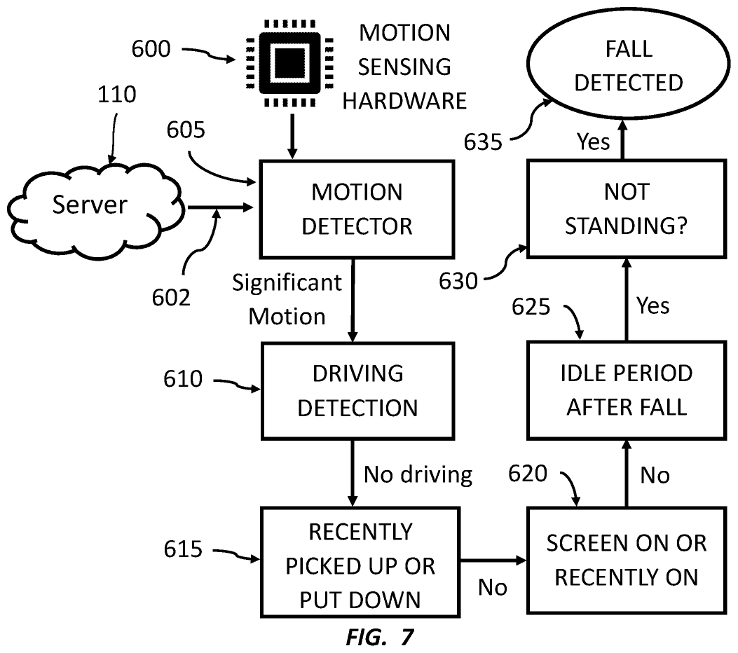

The subsystem for detecting falls is shown in FIG. 7 and consists of multiple steps intended to reduce the rate of false positives. However, the exact sequence and combination of steps could vary as some embodiments might not use every filtering step outlined here. In this embodiment, the motion sensing hardware contains a triaxial accelerometer. However, other forms of motion sensing hardware are possible including but not limited to gyroscopes, infrared sensors, or pressure sensors. Moreover, this particular embodiment is tailored specifically to an Android mobile phone. However, it should be noted that other embodiments may be different phones or different wearable devices including but not limited to watches and wristbands.

Accelerometer data is periodically sampled from the motion sensing hardware 600 and evaluated by the motion detector block 605 in a manner similar to the motion detector unit in FIG. 6. Some embodiments may buffer the accelerometer data in memory before they are processed by the motion detector block. If the embodiment includes an accelerometer, the intensity of the three-dimensional accelerometer (x, y, z) reading could be calculated through the square root of the sum of the squares: \sqrt{x²+y²+z²}, which would be compared to a threshold 602 to determine if the motion is significant enough to indicate a fall (an intensity higher than the threshold would indicate a fall). The threshold value 602 is customizable from the server 110, as caregivers or patients may desire to increase or decrease their fall sensitivity settings to manage the tradeoffs between true positives and false negatives. In this embodiment, the fall sensitivity threshold is configured by the caregiver in the Settings pane of the dashboard 340, where the user can select from several predefined options such as “low sensitivity”, “medium sensitivity”, and “high sensitivity.” After selection is made, the data is stored on the server or database 110 in the form of a number, where it can be retrieved by the motion detector module 605 using a web API.

If the motion detector module 605 determines that no significant motion was found in the recent batch of accelerometer data readings acquired from the motion sensing hardware 600, the present iteration of the fall detection algorithm terminates. In this embodiment, this would occur when no accelerometer value in the buffer has an intensity that exceeds the predefined threshold, or the threshold stored in the server 110 by patient or caregiver selection 602. Otherwise, the system proceeds to the Driving Detection submodule 610.

The objective of the Driving Detection submodule 610 is to ensure that rapid changes in accelerometer data caused by sitting in a moving vehicle are not misreported as falls. The algorithm for determining if the patient is in a vehicle could include further accelerometer-based signal processing. For example, acceleration rates beyond what could be attained by walking or running could be indicative of driving. In alternate embodiments, driving could be detected by establishing a history of GPS data and calculating the speed by dividing the distance between consecutive points by the elapsed time interval. In this particular embodiment, the Driving Detection submodule is implemented by the Google Awareness API, which is a software library available to all Android mobile applications by the Android operating system. Therefore, the implementation of the Driving Detection module is not described in detail.

If it is determined that the patient is not in a vehicle, the system evaluates whether the device was recently picked up or put down 615. If it is determined that the device was put down flat on a surface either shortly before or after the fall, the fall detection algorithms terminates, and no fall is reported. The motivation for this approach to prevent false positives caused by the patient rapidly picking up or abruptly placing the mobile device down. Thus, the subsystem proceeds to the final phase of analysis only if the device has not recently been put on or taken off. In this particular embodiment, the algorithm for determining if the device is placed down is as follows. Several consecutive accelerometer readings are taken immediately before and immediately after the high energy event (possible fall). For evaluating the device orientation before the possible fall at accelerometer sample n, the accelerometer values between n−30 and n−10 are evaluated. For evaluating the device orientation after the possible fall at accelerometer sample n, accelerometer values between n+10 and n+30 are evaluated. The exact buffer size and margins could vary from one embodiment to another. It is determined that the device was placed flat in each cluster of points if the acceleration in the direction parallel to gravity is between 9.6 and 10 m/s, and the acceleration in the other two directions is less than 0.4 m/s for all points within the cluster. Otherwise, it is reported that the device was not placed flat either before or after the possible fall, and the algorithm proceeds.

The fall detection module then evaluates whether the screen of the mobile device is on, as well as how long it has been on or off 620. This is to further filter out false positives caused by a user taking the device out of their pocket abruptly to interact with the device, which could be mistaken for a fall. We do not elaborate on implementation details for this mechanism because in this embodiment, the state of the device display is provided by the Android operating system using an application program interface. In the absence of this interface, the software could periodically query the display hardware to determine its state and register a software timestamp in memory associated with when the screen was last turned on or off. In this embodiment, the present iteration of the fall detection algorithm is cancelled if it is determined that the device display is on or was on within the last two minutes, and a fall is not reported. Otherwise, the algorithm proceeds to the next stage of evaluation.

In the next stage 625, the accelerometer buffer from the motion sensing hardware is analyzed for additional significant motion occurring immediately after the fall. The intuition behind this last layer of screening is that false positives can be further minimized by restricting falls to events associated with a high magnitude motion followed by a period of cessation. Otherwise, it is likely that the patient 100 did not fall, but rather was in a period of intense physical activity. This final stage of evaluation 625 searches the accelerometer buffer derived from the motion sensing hardware 600 for up to a minute after the high-energy event identified by the motion detector 605. As before, this consists of accelerometer-based magnitude thresholding. However, it could also manifest in the form of peak detection or an alternative form of pattern recognition. In one particular embodiment of this approach, the algorithm is terminated if two peaks are found within a minute of the initial fall which have a magnitude of at least 60% of the initial fall threshold reported by the server 110. If it is determined that the possible fall is followed by a period of relatively low activity, the algorithm proceeds to the next step. Otherwise, the present iteration of the fall detection algorithm is terminated without reporting a fall.

The final stage of the algorithm evaluates whether the patient may be standing. In this embodiment, this is evaluated by checking if the magnitude of the acceleration in the axis of the device that would be parallel with the force of gravity if the user was standing (y-axis in this embodiment) was less than 4 m/s for the last five accelerometer readings. If any of the evaluated points are found to have an acceleration value in this direction greater than 4 m/s (it would be close to 9.8 m/s if standing), it is determined that the user is probably not lying on the floor and the present iteration of the fall detection algorithm is cancelled. Otherwise, the system transitions to the Fall Detected state 630 and the caregiver is notified.

### Sleep Quality and Night Motion Subsystem

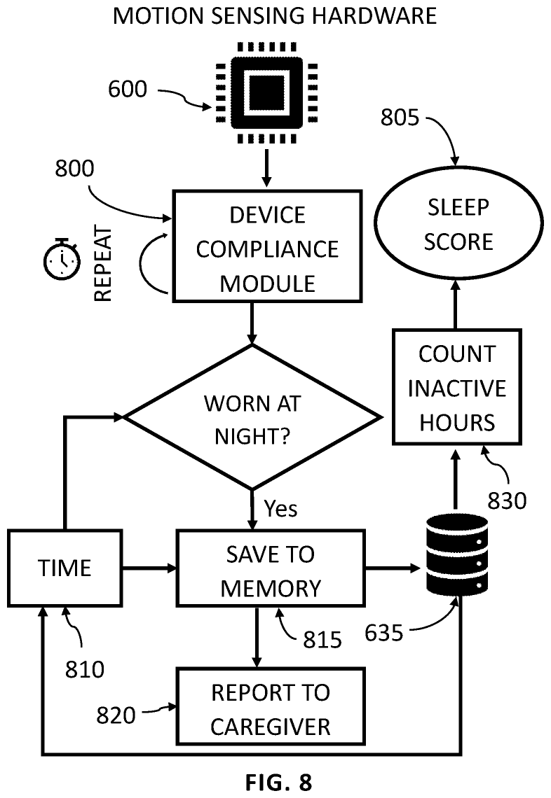

The system can evaluate the quality of the patient's sleep and present a summary to the caregiver in the following day, while also providing immediate notifications if the caregiver is awake during nighttime hours. This subsystem is shown in \*MERGEFORMAT FIG. 8 and begins with the motion sensing hardware 600 which in this embodiment provides regular accelerometer data to the Device Compliance module 800. In this case, the Device Compliance Module 800 is the subsystem described previously, one embodiment of which is summarized in REF_Ref31974519\h\*MERGEFORMAT FIG. 6. First, the Patient Device 105 detects that the patient 100 is in compliance with the device 730 during nighttime hours. If the mobile or wearable device 105 used by the patient is a mobile phone rather than a wrist-worn wearable, patient compliance at night is tantamount to being awake; therefore, the system creates a record in memory 815 indicating the time in which the device was used and proceeds to notify the caregiver.

As there is some ambiguity about the particular hours in which it is appropriate for the patient to be awake, the exact boundaries in which the patient should be sleeping is provided by the server 835. The server contains a record based on the caregiver's selection in the Settings 340 panel of the Caregiver dashboard 115. Thus, when the Device Compliance module 800 reports that the device is used, the current system time reported by the Patient device 105 is compared to the settings loaded from the server 835. If it is found that the Patient 100 is awake at an inappropriate time, this event as well as the associated timestamp is stored in the server/database 835 and a message is sent to the Caregiver device 820. The caregiver receives this message via the Notification subsystem 300, and the Night Motion 365 and Sleep Quality 360 dashboard and detail cards are modified to appropriately summarize the event.

During the following morning, the subsystem queries the database 635 to retrieve a record of when and how long the user was asleep. In this embodiment, this is estimated by counting 830 the hours in which the Patient Device 105 reported a state of Not Worn 735 for the entirety of the hour. If the number of inactive hours does not exceed a particular threshold, which happens to be 6 hours in this particular embodiment, a poor sleep score 805 is assigned to the previous night. The sleep score can be generated in a number of ways, including a linear or non-linear mapping based on the estimated number of hours of sleep. For example, four hours of sleep could be converted to a sleep score of 50, while 8 hours would be associated with a sleep score of 100. However, other embodiments may be rule-based expert systems with strict thresholds and a limited number of discrete sleep quality states. If the Sleep Quality score is low, a Poor sleep message is sent to the Caregiver device 820. The caregiver receives this message via the Notification subsystem 300, and the Sleep Quality 360 dashboard and detail cards are modified appropriately summarizing the event.

### Stress Assessment Subsystem

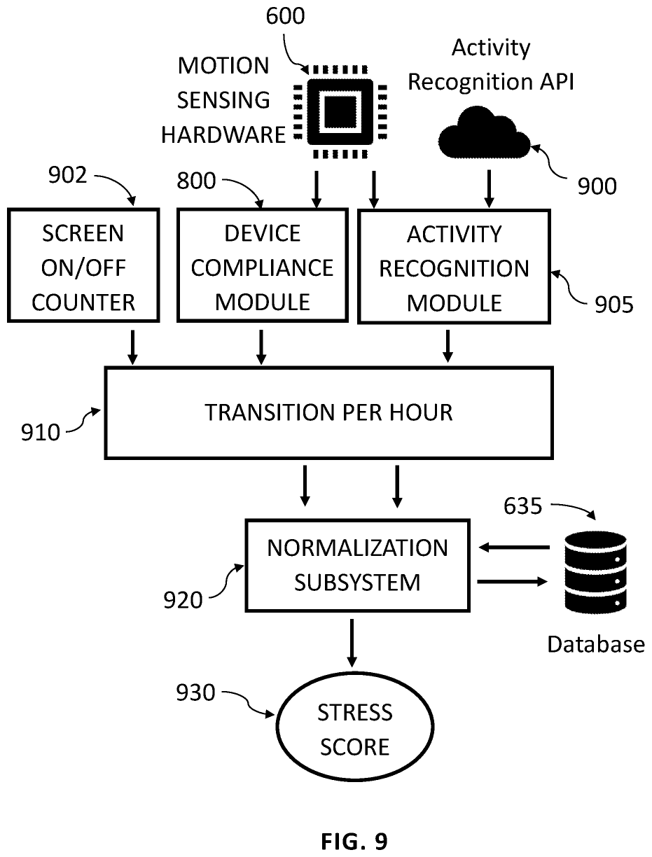

The system is able to estimate the level of stress that the Patient 100 is experiencing, which is reported to the Caregiver 135 in the form of a Stress Score 930 as shown in FIG. 9. Specifically, periods of arousal associated with repetitive motions, pacing, and fidgeting with the device can be captured using the foregoing Device Compliance module 800 described in REF_Ref31974519\h\*MERGEFORMAT FIG. 6, Activity Recognition module 905 and Screen On/Off Counter 902.

The activity recognition module is a subsystem that reports changes in the caregiver's current activity, which could include walking, running, driving, idle, biking, or other forms of physical activity. It could be derived by standard activity recognition algorithms based on accelerometer and gyroscope data from the motion sensing hardware 600 or alternatively by an external activity recognition library such as the Google Awareness library 900 as implemented in this embodiment. Because detecting current activity from a wearable device is prior art, we do not elaborate on implementation details here.

The stress score subsystem runs on the Patient device 105 in this embodiment and counts the number of transitions reported by the Activity Recognition 905 module between an active state (e.g. walking) and inactive state (e.g. idle). When a transition occurs between these two states, a software counter is incremented on the patient device and the number of transitions per hour is updated 910. The intuition behind this heuristic is that repetitive pacing may be associated with anxiety or stress. A user picking up and putting down their phone multiple times within a brief period of time may also be experiencing arousal or stress. Therefore, the module 910 counts the number of transitions between Worn 730 and Not Worn 735 from the Device Compliance module 800 and calculates the number of transitions per hour between these two states. The operation of the Device Compliance module 800 is not repeated here for brevity. Lastly, a record of how often the phone's display has been turned on/off is also counted by another module 902. On many platforms such as the Android and iOS operating systems, the on/off state of the display can be queried directly by an application using a software library. Therefore, the Display On/Off 902 module can count these transitions either by polling or through a software interrupt or callback-based approach, or through other means. Data from all these modules is periodically stored in the database 925, for subsequent analysis.

Information from both subsystems are sent to the Normalization subsystem 920. This block weighs the relative importance of Device Compliance state changes, activity state changes per hour, and screen on/off transitions in calculating the overall Stress Score 930 by means of a linear or non-linear combination. The relative importance of one metric over the other in calculating the final Stress Score 930 is based on a coefficient retrieved from the server 925 configured based on the caregiver's selection in the Settings 340 panel of the Caregiver dashboard 115. Consider an example embodiment in which a caregiver elected to weigh these factors equally. The foregoing subsystems could report that a patient's Device Compliance status has changed twice as often in the last hour compared to their historic average. Similarly, the Transitions Per Hour 915 block following the Activity Recognition subsystem 905 could report that the patient has changed between idle and active states at a rate four times greater than the historic average and the screen has turned on/off at a rate that is average. In this embodiment, the stress score from each module would be weighed equally and averaged using a metric such as harmonic mean, arithmetic mean, or geometric mean. As this stress score likely exceeds the baseline of 1, the caregiver dashboard would be updated accordingly indicating a high level of stress in the recent epoch.

In this particular embodiment, a stress score of over three is considered very high stress, exceeding two is considered high stress, and between 0.5 and 2 is considered within the normal range. However, other embodiments may select different thresholds or alternatively provide a raw numeric value rather than a categorical representation of the stress score. Moreover, some embodiments may elect not to normalize the rate at which the events are taking place to the user historical average but rather do so on the average of other individuals. Other embodiments may forego the normalization step altogether and use fixed thresholds for calculating a stress score from these metrics. Furthermore, of the tree foregoing metrics, some embodiments may calculate a stress score using a subset of these metrics, possibly supplemented by other factors.

If the final stress score calculated during one iteration of this subsystem exceeds the default thresholds described previously, or the predefined threshold selected by the caregiver 135 and stored on the server 925, the patient device 105 sends a message to the caregiver device 115 via the Notification Subsystem 300 indicating high stress, and the Stress 310 dashboard and detail cards are modified appropriately.

The modification to these cards could consist of text change, color change, or any related visual or audio cue summarizing the high-stress event with the objective of bringing the caregiver's attention to this change in status in a manner that is more passive than the Notification subsystem 300. We refer the reader to FIG. 1 for a review of how messages can be passed between the Patient 100 and Caregiver 135. If the Stress subsystem shown in REF_Ref31974536\h\*MERGEFORMAT FIG. 9 does not indicate high stress, a Stress Score is nevertheless calculated and transmitted to the Caregivers 135 through the Caregiver Dashboard 115. Thus, the caregiver will receive messages indicating low arousal in addition to periods of high stress.

### Environmental Monitoring Subsystem

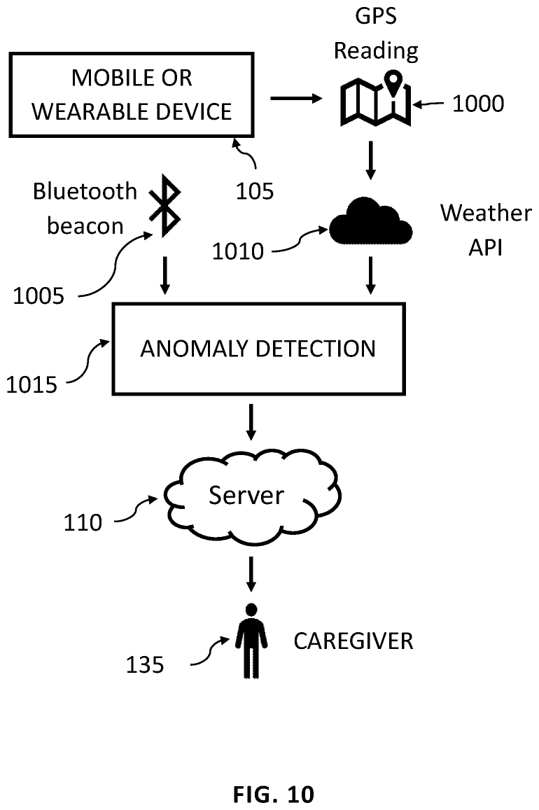

The system can monitor the caregiver's environment for hazards and alert the patient as appropriate through the Caregiver Dashboard 115 and Notification Subsystem 300. The Environment Monitoring subsystem is shown in FIG. 10 and can contain any combination of weather monitoring and/or indoor temperature monitoring functionality. In this embodiment, the external environment monitoring module operates as follows. First, a GPS coordinate 1000 is obtained from the GPS hardware module 205. This information is transmitted to a 3^{rd} party weather service using a web API call to retrieve local weather information. Alternatively, the input to the weather API can be a ZIP code or city of residence manually entered by the caregiver or patient during the registration process. The weather service will return a list of environmental hazards (e.g. county weather alerts) as well as a summary of current and future weather conditions.

If a temperature-enabled Bluetooth beacon 1005 is found in the vicinity of the patient device 105, this temperature reading is collected at regular intervals and stored both on the patient device as well as the server 205. Both the indoor temperature reading as well as the outdoor environment data will be input to the Anomaly Detection 1015 module, which functions as a rule-based system to determine if current environmental conditions are suitable for the patient. In this particular embodiment, the indoor temperature is considered satisfactory if it is between sixty- and eighty-degrees Fahrenheit. The outdoor temperature is not associated with a system-designed threshold. However, extreme or dangerous conditions including but not limited to heavy snow, thunderstorms, winds, flooding, or hurricanes, may result in a weather advisory warning reported by the Weather API 1010, which the Anomy Detection module will recognize. If the Anomaly Detection 1015 module determines that the weather conditions unsatisfactory or unsafe, this information is reported to the caregivers 135 via the server 205.

Urgent time-sensitive notifications related to environmental hazards are immediately delivered to the Caregivers through the Notification Subsystem 300. In this embodiment, the Caregiver would receive an SMS text message and/or a push notification on their mobile phone detailing the hazard. Moreover, the Indoor Temperature 325 and Weather 315 cards would be updated in the Caregiver Dashboard 115 to reflect this emergency. The modification to these cards could consist of text change, color change, or any related visual or audio cue with the objective of bringing the caregiver's attention to the situation. If the caregiver selects or presses the Indoor Temperature 325 and Weather 315 cards in the dashboard, they are presented with a detailed view showing data such as: indoor temperature, outdoor humidity, wind speeds, and weather advisory warnings. In the absence of an emergency, the caregiver can still monitor the indoor and outdoor environment using the Caregiver Dashboard 115.

### Location Subsystem

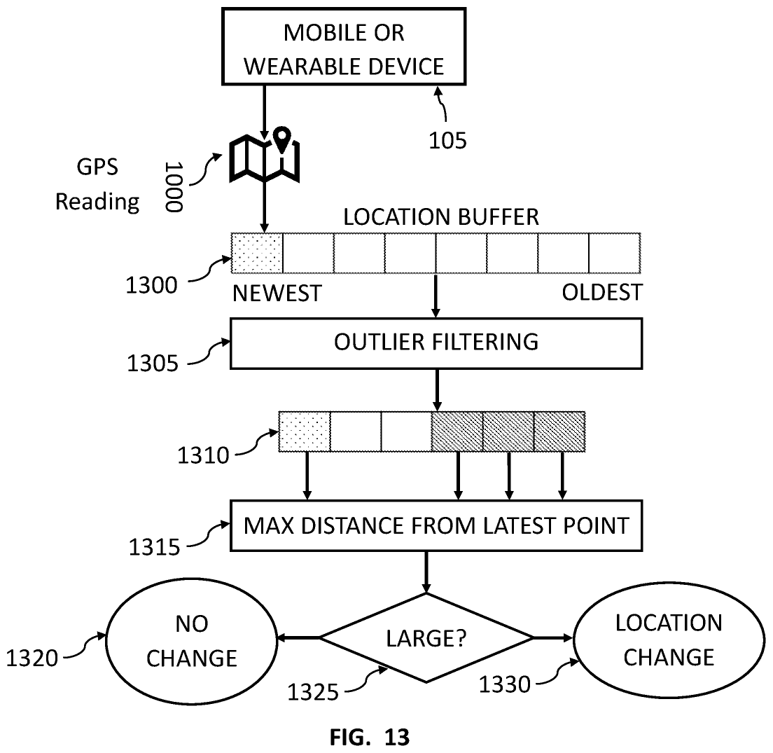

In addition to periodically sending the patient's GPS location to the caregiver dashboard, the Location subsystem is able to alert the caregivers when the patient has moved to a new location by providing the time of the location change as well as the GPS coordinate of the new location. This operation of this subsystem is shown in FIG. 13. Periodically, the GPS 205 of the patient device 105 is sampled for a new GPS coordinate 1000. The operation of this subsystem is independent of the rate at which the GPS coordinates are acquired from the hardware. This coordinate is transmitted to the dashboard and displayed in the Location Card 350 in the form of a map with a dot representing the patient's location. This allows the caregiver to monitor the patient's location from a distance.

Moreover, the patient device maintains a buffer containing a rolling history of GPS data 1300, the exact size of which may vary from one embodiment to another. When a new GPS coordinate is obtained, an Outlier Filtering module 1305 iterates through the entire buffer one coordinate at a time, removing those coordinates that are deemed to be spurious or inaccurate. This is achieved by calculating the distance between each point n and the previous point, n−1, as well as the following point, n+1. If the distance between n and either of its immediately neighbors is significantly larger than the distance between the two neighbors (n−1 and n+1), the coordinate is filtered out. In this particular embodiment, a coordinate is filtered out if the neighboring points are within 40 m of each other, but the distance between the point and either of its neighbors exceeds 80 m. However, other values for these parameters can be used in alternate embodiments. Furthermore, alternate embodiments may compare a point with multiple adjacent points instead of the immediate neighbors. Moreover, it should be noted that alternate embodiments of the location subsystem may forego the Outlier Filtering module altogether.

After the Outlier Filtering module 1305 removes unreliable GPS coordinates from the buffer, another module 1315 uses the new buffer 1310 to calculate the distance between a recent coordinate and several older GPS readings from the buffer. In some embodiments, the recent coordinate may not necessarily be the most recent one, as there may be insufficient location context to deem that particular coordinate reliable. In this embodiment, the GPS buffer contains the last hour of GPS history data and this module 1315 calculates the distance between the latest GPS reading and all GPS readings obtained between 30 minutes and one-hour prior. If the maximum distance between the recent location and any of these historic locations exceeds a predefined threshold that would constitute location change, the caregiver is notified through the caregiver dashboard and through the Notification Subsystem 300.

### Machine Learning Subsystem

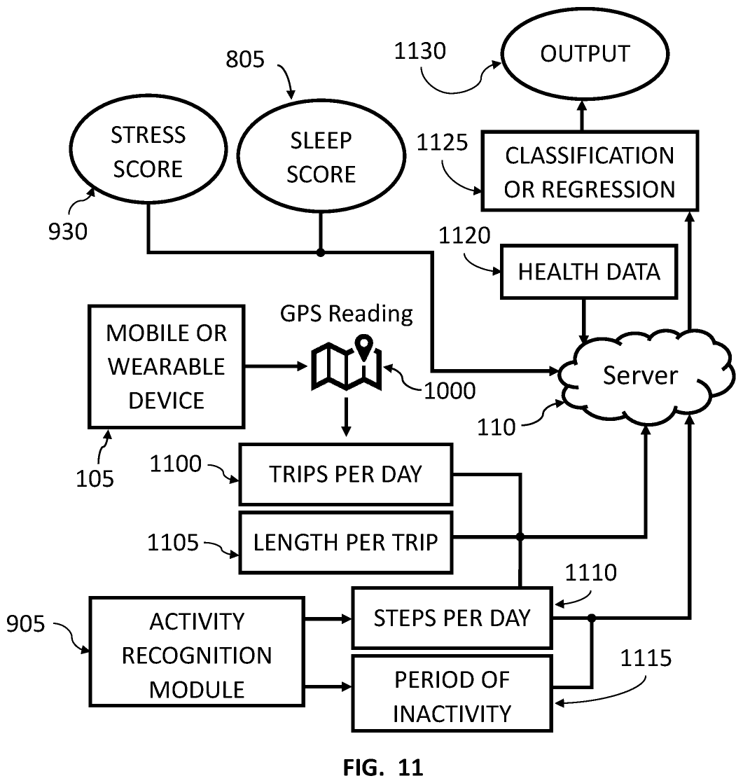

The Machine Learning subsystem is a flexible framework for screening and tracking of various physiological and mental health disorders using the foregoing sensing modalities. One embodiment of this system is shown in FIG. 11. During initial setup and account registration, patient demographic information and health data 1120 are saved to the database on the server 110. This information is entered by manual entry on the caregiver dashboard 115 and could include: age, height, weight, gender. It would also include a list of conditions associated with the patient (e.g. depression, anxiety, Alzheimer's) available for entry both in free-text form and as a dropdown list of common physical, mental, and psychological conditions.

After this information is entered, normal device use longitudinally aggregates a variety of metrics about the patient's health and wellness as derived from the wearable sensors of the Patient device. This information is processed and saved on the server 110 for further analysis alongside the information gathered about the patient's existing health conditions acquired during the user registration process 1120 described previously. Thus, it is possible to associate the self-reported physiological/psychological conditions the health data collected during continuous use of the device.

While the exact health metrics used by the Machine Learning Subsystem could vary, this embodiment include the aforementioned Stress Score 930 calculated by analyzing pacing and Device Compliance transitions, the Sleep Score 805 calculated based on the number of hours at night the device has not been worn, daily and weekly step counts. This information is supplemented with additional metrics inferred data acquired from the GPS module 100 of the mobile/wearable device 105. These metrics include the number of times the patient 100 has left their home over the last week, and the average durations of these trips. Consider a patient who leaves their home for two hours per day, five days of the week. This individual would have a Trips Per Day score of (5/7) and a Length Per Trip score of 2.

The system calculates the number of times the patient has left their home over the last week by periodically calculating the distance between the patient's current GPS location 1000 and the location obtained from the GPS in the previous night when the device was not worn. If the distance exceeds several hundred meters, the system registers that the user has been away from home in the last epoch and a counter is incremented. The counter is not incremented again until the user returns home and leaves once more.

Calculating the average duration of trips in which the patient leaves their home is a minor extension of the foregoing system to detect how often the patient leaves their home. A software timer is run the first time the device finds a GPS location significantly further than the location the patient spent the previous night. The next time the patient returns to their home and the latest GPS location is near the location of the home, the timer is stopped. Thus, the timer represents a running total of the amount of time the patient spent outside their home. The value of this timer can be divided by the number of trips to calculate the average length of each trip.

The Activity Recognition Module 905, which as mentioned could come either from the motion sensing hardware 600 or from a 3^{rd}-party API such as Google Fit, would provide additional fitness information such as Steps Per Day 1110 and the Period of Inactivity 1115 metric. The Steps Per Day 1110 metric is an average daily step count since the last evaluation of the algorithm and is readily available on most smartphone platforms through standard APIs. Alternatively, many libraries and frameworks for calculating step count from motion sensing hardware 600 data are available. The Period of Inactivity 1115 metric calculates the total number of hours over the course of the last week in which the patient did not walk at least thirty steps, though the exact number could vary from one embodiment to another. Additional information would be derived from other wearable sensors. If the device contains an optical sensor such as a pulse oximeter, the patient's SPO₂ and resting heart rate would be acquired and saved as well.

Lifestyle-related data from the GPS 1100, 1105, fitness 1110 and inactivity 1115 information from the Activity Recognition module 905, stress information 930 and the sleep score 930 all form the basis of the feature set used by the machine learning subsystem. However, it should be noted that other embodiments could employ additional features or some subset of the aforementioned features. As one familiar with standard practices in machine learning will recognize, the objective of a typical machine learning system is to use the input feature set to predict one or more outcomes. In this case, the outcomes would be the health metrics 1120 associated with the patient which are stored on the server. The inputs would be behavioral data derived from the various wearable sensors on the patient device. Thus, the classification/regression module 1125 will train a statistical model to correlate health outcomes with the behavioral data collected by the system, using a dataset of potentially thousands of individuals generated from the entire userbase of the health monitoring system. After each evaluation, the classification/regression module will supplement its internal dataset with data from the current patient being evaluated. This data will be stored on the server 110 and will improve the accuracy of future iterations of this subsystem.

Consider a scenario in which most individuals who register their anxiety disorder in their health data 1120 are found to have a high stress score 930 and poor sleep score 805. Thus, the statistical model 1125 will learn to associate these characteristics with this health outcome through classification (assigning a discrete condition to the patient such as “Anxiety” or ‘No Anxiety”) or regression (assigning a probability or severity score, such as an Anxiety score of 0.9) during the training process that will occur nightly and include all available patient data on the server. If a particular user's sleep score improves in a following month, the regression module in the subsystem 1125 could output 1130 that the similarity score between this individual and the typical individual with anxiety has decreased. For example, their status may be assigned from “Anxiety” to “No Anxiety”. Alternatively, their anxiety score could have decreased from 0.9 to 0.7.

Similarly, a patient who does not self-report anxiety that is exhibiting characteristics associated with this cohort will be notified of their elevated risk from the system output 1130, that will in turn interface with the caregiver dashboard 115 and Notification Subsystem 300. The Machine Learning Subsystem therefore presents a flexible, scalable approach to identify and track various mental and physiological disorders through the patient's behavior, physiological data, interaction with their mobile/wearable device 105.

The details of the operation of the Classification/Regression block 1125 could differ entirely from one embodiment to another. As those versed in standard practices of machine learning will recognize, such statistical learning tools could include but not be limited to decision trees, support vector machines, logistic regression, deep learning, and random forest. The aforementioned techniques are all capable of associating arbitrary input variables (e.g. stress score, sleep score) with discrete (e.g. “depression” or “anxiety”) or continuous (e.g. “depression=0.9, anxiety=0.3”) outputs. A variety of libraries, APIs, and frameworks are readily available for implementing statistical learning algorithms on mobile/wearable devices, and there is no particular approach that must be employed.

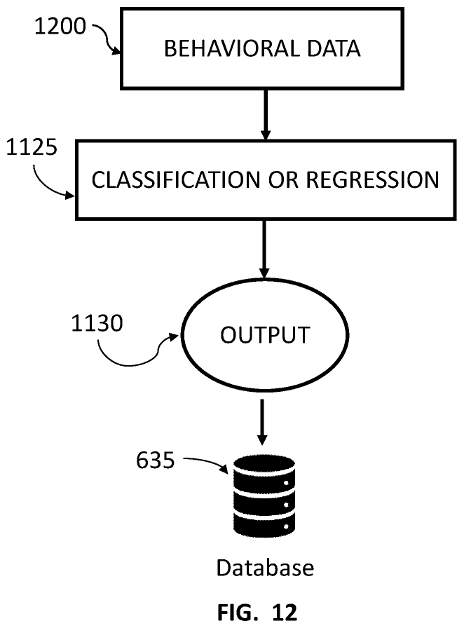

A more simplified representation of the Machine Learning subsystem is shown in FIG. 12. Periodically, the behavioral data acquired from the patient 1200 is input into the Classification/Regression block 1125, which has been pre-trained to output one or more associated health conditions in the form of a discrete or continuous output 1130 that is saved to the database 635. The behavioral data in this case is a vector of data gathered about the patient from the various wearable sensors on the device (e.g. stress score, sleep score, and other metrics outlined previously). The details of the method in which the Classification/Regression module is trained are standard practice in the field, and are based on the behavioral data as well as the associated health data 1120 manually entered by the user that functions as a gold standard for the accuracy of the device.

### CONCLUSIONS, RAMIFICATIONS, AND SCOPE

Accordingly, the reader will see that the proposed system is a remote health monitoring platform for the individuals of limited independence, designed to effectively provide continuous real-time feedback and assurances of the patient's well-being as informed by multiple disparate sensing modalities including audio, motion sensors, proximity sensors, GPS signals, and other sources of data. Thus, the system is designed not only for the safety of the patient by providing immediate notifications of deleterious outcomes, but also serves to reduce the psychological burden of fulfilling the obligations of the caregiver through continuous assurances of the individual's welfare.

Moreover, the system includes of a novel approach for the detection of falls from a wearable sensor that considers a combination of factors which may include the recently of the device being worn or taken off, if the wearer is in a vehicle, and if the event is followed by a period of cessation, in order to reduce the rate of false positives. The mechanism also incorporates an additional sensitivity parameter configurable from the cloud, enabling the caregiver to further personalize their desired tradeoff between accuracy and sensitivity.

Furthermore, the platform includes a novel approach to detect device adherence in a time-sensitive manner by considering both device motion as well as the orientation of the device with respect to gravity, for highly accurate and responsive characterization of usage. Disambiguation between scenarios in which the device is not worn, and if the device is worn but the wearer is idle for an extended period of time, could otherwise cause a very large latency and significant inaccuracy. By considering the device orientation (flat) as an override, the proposed system is capable of detecting if a device is not worn more quickly than state of the art approaches.

The system also presents a framework for detecting motion at night, as well as characterizing the number of hours of sleep the individual received using data from the motion sensing hardware included in the wearable sensor. This information is consolidated into a single Sleep Score and reported to the caregiver. Additionally, the platform features a method of characterizing stress by analyzing the rate in which the device adherence subsystem transitions from one state to another, combined with the rate of activity state transitions between active and idle. These two parameters are combined into a single Stress Score with a coefficient configurable from the cloud based on their unique understanding of the patient's expected pattern of behavior.

The system represents a framework to holistically profile an individual's wellness including stress, sleep quality, fitness, and longitudinal lifestyle changes. As the mobile system is used in the field, a large-scale database of behavioral data is generated from the userbase, providing a flexible platform for the longitudinal tracking and screening of a variety of mental, neurological, and physiological disorders. Upon entering demographic information, and a variety of behavioral data is derived from the patient's device and input into a machine learning module to find correlations between the wearer's lifestyle, habits, and fitness, and other users who self-report a condition such as depression or anxiety.

In the foregoing specification, embodiments have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction.

What is claimed is:

1. A system for the remote monitoring of an individual of limited independence by one or more caregivers, comprising:
(a) a mobile or wearable device to be worn by said individual that includes a processor, one or more communication interfaces, one or more sensors coupled to the processor, and memory containing computer-readable code which when executed by a processor, collects a multitude of health, environment, lifestyle, and safety metrics from said individual and transmits them to a server or database;
(b) a dashboard accessible by one or more caregivers in the form of a mobile or web application that loads data from said server or database, wherein real time notifications and alarms of said individual's status can be received in the form of automated calls, mobile push notifications, text messages, or other means, and additionally, real time and historic visualizations and analysis of the health, behavior, lifestyle, and safety of said individual can be viewed, which comprise a multitude of the following: stress detection, device adherence detection, location, fall detection, physical activity detection, daily step totals, heart rate, weather hazard detection, indoor temperature detection, and sleep tracking.

2. The system of claim 1, wherein said user's collected health, behavior, lifestyle, and safety metrics are used to predict the existence and severity of physical and mental illnesses, comprising:
(a) a machine learning classifier periodically trained to correlate said metrics collected from the userbase and stored in said database, with self-reported physiological and psychological conditions collected during account registration;
(b) the ability of said machine learning model to predict and report health conditions and severity scores of such conditions using classification and regression based on said metrics, and optionally notify the caregiver through said dashboard.

3. The system of claim 2, wherein the inputs to the machine learning model include the average number of times per day that the individual has left their home, the total length of time spent outside the home, the average length of time between when the user left their home and when they returned, or any combination therein.

4. The system of claim 1, wherein said individual's stress level is estimated based on the frequency of their interactions with said device and patterns in said individual's movement history and reported to the caregiver.

5. The system of claim 4, wherein said individual's stress level is calculated with one or more of following metrics: the rate at which the device state has recently transitioned between worn and not worn, the rate at which the user has transitioned between walking and idle, and the rate that the mobile device's display has recently been turned on and/or off.

6. The system of claim 1, further comprising a temperature enabled beacon placed in the vicinity of said individual; the temperature of the individual's surroundings being collected and transmitted to said caregiver for real-time tracking and notifications of hazardous conditions in the form of automated calls, mobile push notifications, text messages, or other means.

7. The system of claim 1, wherein outdoor temperature conditions or weather alerts in said patient's vicinity are acquired using a web API and transmitted to said caregiver's dashboard.

8. The system of claim 1, wherein the caregiver is remotely alerted of significant changes in the patient's location using data from global positioning system hardware included in said mobile or wearable device.

9. The system of claim 8, further comprising a location filtering mechanism that uses the distance between a point and its neighbors, and the distance between the said neighbors, to detect and remove inaccurate readings, whereby the accuracy of the location change functionality is improved.

10. A method for detecting if a mobile or wearable device is used or worn by interpreting the existence of recent motion of sufficient intensity from motion detector hardware as indication of device adherence, and by further interpreting the absence of significant motion for an extended period of time as indication that the device is unused or not worn.

11. The method of claim 10, wherein said motion detector hardware is an accelerometer and said device is immediately detected to be not used/worn when placed down on a flat surface, determined by comparing the force of gravity to the acceleration of the axis of the accelerometer that would be parallel to gravity if placed down, whereby detection that the device is not used occurs with less delay than would be otherwise possible.

12. The method of claim 10, wherein said individual's sleep quality is estimated by counting the number of hours during nighttime in which the device was unused, whereby the caregiver is notified of said individual's sleep quality.

13. The method of claim 10, wherein the caregiver is notified if the patient is awake during expected night-time hours by detecting motion in the device using motion sensing hardware at any time during this period.

14. A method for detecting if the wearer of a mobile device with a motion sensor has fallen down by identifying a high magnitude motion event from a hardware motion sensor.

15. The method of claim 14, wherein the user is reported to have fallen only if said high magnitude motion event is immediately followed by a period of low activity or inactivity as determined by said motion sensor, whereby the rate of false positives is reduced.

16. The method of claim 14, wherein the rate of false positives is reduced by detecting if the wearer of the device is in a vehicle and by either not reporting a fall in these scenarios or otherwise adjusting the operation of the system to reduce said false positives with this information.

17. The method of claim 14, wherein the rate of false positives is reduced by detecting if the status of the device changed between worn and not worn approximately the same time as said high magnitude motion event, and by either not reporting a fall in these scenarios or otherwise adjusting the operation of the system to reduce said false positives with this information.

18. The method of claim 14, wherein the rate of false positives is reduced by detecting if the device display was on/off at the same time as said high magnitude motion event or was turned on/off shortly prior, and by either not reporting a fall in these scenarios or otherwise adjusting the operation of the system to reduce said false positives with this information.

19. The method of claim 14, wherein the rate of false positives is reduced by estimating if the user is standing upright shortly after said motion event by measuring the magnitude of acceleration in the axis that would be closest to parallel with gravity if the user was standing, and by either not reporting a fall in scenarios when said magnitude is not significantly less than the gravitational acceleration, or otherwise adjusting the operation of the system to reduce said false positives with this information.

20. The method of claim 14, wherein the minimum magnitude of said motion event is remotely configurable by said caregiver or said patient whereby they can manage the sensitivity of the fall detection system.
